(19) United States
(12) Patent Application Publication
(10) Pub. No.: US 2026/0315105 A1
(43) Pub. Date: Oct. 15, 2026

(54) MEDICAL ACCESS SYSTEM

(71) Applicant: EBNET MEDICAL GMBH, Schwerin (DE)

(72) Inventors: Jens EBNET, SCHWERIN (DE); Sven LAHME, PFORZHEIM (DE); Werner SCHWARTZ, RUHPOLDING (DE)

(21) Appl. No.: 19/101,111

(22) PCT Filed: Aug. 15, 2023
(86) PCT No.: PCT/EP2023/072452

§ 371 Date: Feb. 4, 2025

(30) Foreign Application Priority Data
Aug. 16, 2022 (DE) 10 2022 120 628.9

(51) Int. Cl. A61B 17/34; A61B 1/00; A61B 1/313; A61B 17/22

(52) U.S. Cl. CPC A61B 17/3423; A61B 1/00154; A61B 1/313; A61B 17/22; A61B 17/3496

(57) ABSTRACT

A medical puncture system for carrying out a puncture on a patient, and is suitable particularly for carrying out a minimally invasive percutaneous nephrolitholapaxy (mini-PCNL).

### FIELD OF THE INVENTION

The invention relates to a medical puncture system which may be used for punctures of any type, and is particularly for carrying out a minimally invasive percutaneous nephrolitholapaxy (mini-PCNL).

### BACKGROUND

Mini-PCNL is a urological surgical method for removing kidney stones. Although instrument miniaturization has already made it a comfortable and uncomplicated procedure, carrying it out requires some working steps that also partly need to be carried out under radiological and sonographic monitoring.

Initially, the calyceal system of the kidney is punctured with a thin needle under ultrasound monitoring. Then, after installation of a guide wire through the needle and retraction of the latter, under radiological monitoring the puncture channel is widened with a dilator threaded over the guide wire so that a so-called Amplatz shaft can then be introduced through the dilator. After removal of the dilator, a specialized endoscope, a so-called nephroscope, is pushed forward through this Amplatz shaft into the calyceal system of the kidney. A laser fiber, which is used for lithotripsy of the kidney stones, is guided through the working channel of the nephroscope. The resulting stone fragments are subsequently washed out between the Amplatz shaft and the nephroscope by the permanent irrigation flow in the calyceal system of the kidneys. This conventional way of carrying out a mini-PCNL therefore requires several different instruments, which need to be provided separately and are used in succession.

DE 10 2009 060 921 A1 discloses a puncture needle system for puncturing an organ, in particular a kidney, which contains optics in the puncture needle tip. The known puncture needle system is designed only for relatively small inner lumens of the working shaft, with the result that the outer diameter of the working shaft should be relatively small (less than 6 Fr) and further bougienage of the access opening should be avoided. Only a relatively small inner lumen is therefore available for carrying out the work. If the diameter of the working shaft is not sufficient to receive a device for disintegrating the stone, which for example is used for mechanical lithotripsy or operates with laser energy, in the case of the known puncture needle system it is first necessary to extract the working shaft from the channel, insert a guide wire and introduce a larger working shaft having an obturator for bougienage of the access along this guide wire.

Such a procedure is relatively time-consuming, requires very meticulous work by the operator and entails an increased stress on the patient.

### SUMMARY

The object of the invention is to provide an improved puncture system, which avoids the disadvantages explained above and therefore improves the safety of the medical intervention.

This object is achieved by a medical puncture system having a shaft unit, a dilator unit and a puncture needle unit for carrying out a puncture on a patient, having the following features:
a) the shaft unit has an outer tubular body with an inner working lumen, and a first grip section which is adapted for manual handling of the shaft unit and is connected at a patient-remote section to the outer tubular body,
b) the dilator unit has an inner tubular body with an inner puncture lumen, and a second grip section which is adapted for manual handling of the dilator unit and is connected at a patient-remote section to the inner tubular body, the inner tubular body being guidable through the working lumen of the outer tubular body,
c) the puncture needle unit has a puncture needle and a third grip section, which is adapted for manual handling of the puncture needle unit and is connected at a patient-remote section to the puncture needle, the puncture needle being guidable through the puncture lumen of the inner tubular body.

The puncture system according to the invention makes it possible to carry out minimally invasive interventions, in particular a mini-PCNL, with a single integral tool system in which all required elements are integrated and the operator does not need to resort to additional instruments, i.e. it is not necessary to change between individual instruments. The operator can carry out the complete puncture as far as the precise installation of the outer tubular body having a comparatively large lumen smoothly and with little stress on the patient. The medical puncture system assists the operator by its design features in respect of safe, error-free and efficient work.

The outer tubular body may be dimensioned in such a way that a device for disintegration of the stone and aspiration of the stone fragments can be guided through this outer tubular body. The outer tubular body may, for example, be configured as an Amplatz shaft. By the integrated dilator, the necessary widening of the puncture point can be carried out in one working operation.

The terms “puncturing” and “puncture” are to be understood in the medical sense. “Puncturing” refers to the entry of a puncture needle into the hollow body in such a way that the puncture needle penetrates through the tunic of the hollow body to be punctured.

Those parts which are arranged at the remote (distal) end of the puncture system from the viewpoint of the user, and therefore in the vicinity of the punctured region of the patient, are regarded as a near-patient section of parts of the puncture system. Correspondingly, patient-remote sections are arranged at the proximal end of the puncture system from the user's viewpoint, i.e. further away from the punctured hollow body. When the word “or” is used in what follows, possible alternatives are indicated but combinations of the features or configurations separated by “or” are in principle also explicitly possible. In the sense of the present invention, an indefinite article “a” cannot be understood as a numeral. Thus, for example, when a constituent part is referred to, this is to be interpreted in the sense of “at least one constituent part”.

The invention also provides an improved system in the sense of a general puncture system. In principle, with the puncture system according to the invention it is likewise possible advantageously to puncture all body cavities and interstitial body spaces and all anatomical and morbid structures that are intended to be punctured, and to carry out corresponding interventions.

Components of the improved puncture system may in principle also be combined with all known intervention, puncture and catheter systems or else be used as independent products.

The term “puncture system” will be adopted below, although it has a wider meaning in the sense of a general puncture system with which not only mini-PCNL may be carried out.

With the puncture system according to the invention, interventions may in principle be carried out in and on all body cavities and interstitial body spaces and all anatomical and morbid structures, examples which may be mentioned being the trachea, pleural space, abdomen and pelvic space, stomach, intestine, gallbladder, urinary bladder and cerebrospinal fluid space. In addition, intervention may be carried out on morbid structures such as abscesses in and on the patient.

In what follows, the term “patient” includes all living beings of all age groups and genders. Applications in technical fields and in and on all objects and structures are likewise explicitly possible, for example in and on reservoirs, containers, cavities, extensible materials and in or on pump, tube, pipe and port systems.

All components described may be employed singly or multiply on a puncture system or also, independently of the latter, on/in other products or entirely independently. Various features of different components are also freely combinable, and features of particular components may also be used on other components without this being explicitly mentioned. All components and features may in principle be employed inside and outside a patient.

The puncture system according to the invention is distinguished in that it combines all components necessary for carrying out a PCNL, in particular a mini-PCNL, into one instrument. All components are connected to one another and releasable from one another on the grip region. The user can activate the connecting and release steps only in a particular sequence on the grip region. This ensures a safe sequence of the individual process steps and thus prevents the risk of accidental incorrect actuation of the individual components.

The first, second and third grip sections are each shaped in a way that is particularly favorable for gripping, i.e. ergonomically shaped, so that they assist simple and secure gripping by the user. This has the advantage that the user does not need to grip the individual parts of the puncture system at places not actually intended therefor, for example at Luer lock connections, but is provided with defined grip sections which, because of their configuration favorable for gripping, stand out visually from the other parts of the puncture system and can therefore easily be discerned as grip sections by the user. This is also conducive to working intuitively with the puncture system according to the invention.

The first, second and/or third grip sections may, for example, have a hyperboloid shape. For example, the curvature of the hyperboloid shape of the second grip section may be less than the curvature of the first and/or third grip section. The first, second and/or third grip section may also be configured as another rotationally symmetrical body. There may also be grooves and/or pimples, or other surface structuring, on the outer surface of the first, second and/or third grip sections in order to increase the grip.

Some or all grip sections may at least partially consist of a material that increases the frictional resistance, or be coated with at least one such material. This prevents the finger of the user from inadvertently slipping. The grip region may also consist fully or partially for example of an elastomer, for example rubber or silicone, or of any other material that is softer than the rest of the material of the puncture system.

Some or all grip sections may also consist of at least one antimicrobially effective material, or be coated with at least one such material.

Some or all grip sections may also consist of at least one material that has particularly hydrophobic properties, or be coated with at least one such material. Properties in the sense of a lotus effect or similar to a lotus effect may also be envisioned.

Some or all grip sections may at least partly be configured in a different color than the other components of the puncture system, for example in a green, yellow or red color. In particular, the grip sections may be configured in different colors. This is intended to remind the user of the colors of traffic lights and may indicate the sequence of the operating steps. In addition, the sequence of the operating steps may be displayed by numbering or labeling in general, for example numbering or other labeling of the grip sections. Special patterns are likewise possible in the grip region, for example patterns resembling crosses or arrows.

It is also conceivable that regions of the puncture system which must not be touched, for example in order to prevent contamination with germs, are configured at least partly in a red color or other conspicuous color.

The color “yellow” may also be envisioned at least partially in regions which ought not to be touched, but which it is nevertheless permissible to touch in special situations. The color “green” may then be used for the regions which may be touched.

Moreover, strongly luminous or fluorescent colors may in particular also be envisioned.

It is also conceivable that some or all grip sections are initially covered by an adhering structure, for example a film, which may be removed before, during or after the puncture process. The features explained for the grip sections may also be implemented individually or in combination on one or more other components of the puncture system.

In a further advantageous development, the puncture system is configured to be partially, predominantly or completely transparent so that flows of fluid/urine/blood can be identified directly. The puncture needle may also be configured to be transparent if the material properties allow this.

According to one advantageous configuration of the invention, the first grip section can be releasably coupled via at least one form-fit coupling element to the second grip section by a form fit and/or the second grip section can be releasably coupled via at least one form-fit coupling element to the third grip section by a form fit. This has the advantage that that the first grip section can on the one hand be coupled reliably to the second grip section, but when required can also be released therefrom so that the corresponding subunits of the puncture system can be removed after carrying out a working step of puncture on the patient. Likewise, the second grip section can be coupled reliably to the third grip section but also released therefrom when required.

According to one advantageous configuration of the invention, the first, second and third grip sections form a homogeneous grip section for gripping and manually handling the puncture system when they are coupled to one another via the form-fit coupling elements. The homogeneous grip section formed in this way forms a kind of handle of the puncture system. The first, second and third grip sections may respectively form a subsection of such a handle. The homogeneous grip section may, for example, be configured homogeneously in the sense that the first grip section merges into the second grip section without a perceptible transition or with a smooth transition. The second grip section may also merge into the third grip section without a perceptible transition or with a smooth transition. The first grip section may directly adjoin the second grip section. The second grip section may directly adjoin the third grip section.

According to one advantageous configuration of the invention, the second grip section coupled by a form fit to the first grip section can be released from the first grip section by a rotational movement in relation thereto and/or the third grip section coupled by a form fit to the second grip section can be released from the second grip section by a rotational movement in relation thereto. This reliably prevents accidental release of one grip section from the other grip section. For the release, the user must carry out a defined rotational movement of a grip section in relation to the neighboring grip section. The rotational movement required for the release of one grip section from the other grip section may, for example, be a rotation in an angle range of from 90 to 180 degrees. In particular, a screw thread may be avoided for producing the form-fit coupling, which has the advantage that multiple rotation, for example through an angle of more than 360 degrees, is not necessary for the release of one grip section from the other grip section.

The second grip section may, for example, be released from the first grip section by a rotational movement about the longitudinal axis relative thereto. The form-fit coupling between the first and the second grip sections may be configured in the manner of a bayonet connection. The third grip section may also be released from the second grip section by a rotational movement about the longitudinal axis relative thereto. The form-fit coupling between the second and the third grip sections may be configured in the manner of a bayonet connection.

According to one advantageous configuration of the invention, the puncture needle is longitudinally displaceable in relation to the inner tubular body, the puncture needle unit being withdrawable from the dilator unit in a direction facing forward from the near-patient end of the puncture system. In order to withdraw the puncture needle unit, the form-fit coupling between the second and the third grip sections first needs to be undone.

According to one advantageous configuration of the invention, the inner tubular body is longitudinally displaceable in relation to the outer tubular body, the dilator unit being withdrawable from the shaft unit in a direction facing forward from the near-patient end of the puncture system. In order to withdraw the dilator unit, the form-fit coupling between the first and the second grip sections first needs to be undone.

In such an advantageous configuration, for example, the puncture may be carried out in such a way that initially only the puncture needle is guided with its tip forward to the desired position inside the body of the patient. The form-fit coupling between the second and the third grip sections is then released. The still connected unit consisting of the dilator unit and the shaft unit can now be pushed forward. It is guided over the puncture needle. The puncture needle therefore acts as a guide wire. The opening is widened by the dilator unit so that the shaft unit can also be pushed forward with it. Once the unit consisting of the dilator unit and the shaft unit is placed at the proper location, the puncture needle may be withdrawn, i.e. the puncture needle unit is extracted from the inner tubular body. The form-fit coupling between the first and the second grip sections may then be released and the shaft unit can be pushed forward and placed at the proper location. The dilator unit may subsequently be removed by extracting it from the outer tubular body. Only the shaft unit with the outer tubular body having a relatively large lumen therefore remains on the patient. For example, a lithotripter may be guided directly through this outer tubular body.

According to one advantageous configuration of the invention, the puncture system has a securing mechanism, which ensures that the dilator unit can be withdrawn from the shaft unit only when the third grip section is decoupled from the second grip section and/or the puncture needle unit is withdrawn from the dilator unit. This assists the operator in a safe working practice. Because of its design, the puncture system precludes the possibility that the dilator unit can be wrongly extracted together with the puncture needle unit or that the shaft unit can be pushed forward without the dilator unit.

According to one advantageous configuration of the invention, the form-fit coupling between the first and the second grip sections is secured via the securing mechanism, the securing mechanism having a secured state and an unsecured state, release of the first grip section from the second grip section being prevented in the secured state and release of the first grip section from the second grip section being enabled in the unsecured state. This allows simply designed production of the securing mechanism together with a reliable function. In particular, the securing mechanism may be produced by component parts that interact purely mechanically with one another.

According to one advantageous configuration of the invention, the puncture needle unit has at least one securing element, which blocks the securing mechanism in the secured state which prevents the securing mechanism from being put into the unsecured state. The securing element may, for example, project from the third grip section in the direction of the first grip section and extend in the longitudinal direction predominantly or fully through an inner chamber of the second grip section.

According to one advantageous configuration of the invention, the third grip section is arranged further away than the second grip section from the near-patient end of the puncture system, at least when the puncture needle is guided through the puncture lumen of the inner tubular body.

According to one advantageous configuration of the invention, the second grip section is arranged further away than the first grip section from the near-patient end of the puncture system, at least when the inner tubular body is guided through the working lumen of the outer tubular body.

According to one advantageous configuration of the invention, the inner tubular body has a dilation section in a near-patient region, which tapers on the outer circumference toward the near-patient end of the puncture system. The dilation section may, for example, follow on directly from a hollow-cylindrically shaped part of the inner tubular body. The dilation section may for example have an outer contour configured linearly, progressively or degressively in the shape of a ramp. The difference between the outer diameters of the inner tubular body and of the outer tubular body is relatively small, for example less than 50% of the outer diameter of the inner tubular body.

According to one advantageous configuration of the invention, the puncture needle is configured as a hollow needle and there is an opening in the region of the third grip section, through which liquid can be conveyed outward through the puncture needle from the near-patient region of the puncture needle. This has the advantage that correct progress of the puncture and correct placement of the puncture needle tip can be checked with the aid of liquids emerging from the opening. There may for example be a tube connection at the opening, for example a Luer lock connection.

The puncture needle may have a tip at the near-patient end, for example in a conical shape or in the form of a bevel.

Materials for the puncture needle may be metal materials, in particular stainless steel or in general metal alloys. The puncture needle may, however, also consist of a plastic material. It may also fully or partially be formed in lightweight design or from at least one material that is resorbable/readily soluble in urine, blood, infusion solutions or other fluids, for example a sugar or a salt, for example in the region of the puncture tip. Magnesium may also be used. The puncture needle may likewise advantageously be developed further with at least one sensor, for example in the region of the puncture tip.

According to one advantageous configuration of the invention, the puncture needle has at least one optical component at least in the near-patient region, through which light can be shone toward the patient and/or images can be recorded. The optical component may, for example, be coupled to a display device arranged separately from the puncture system or alternatively fastened to the puncture system. For example, image information recorded by the optical component may be reproduced on the display device. In this way, the operator can visually check correct progress of the puncture and proper placement of the puncture needle tip.

It also conceivable that a commercially available smartphone or another device that communicates with the optical component and/or with other components of the puncture system may be used as a display device. Communication may also take place wirelessly via waves/electromagnetic fields, for example.

The optical component may for example have an electrical light source, an optical fiber and/or a camera. If the puncture needle is configured as a hollow needle, the optical component may for example be arranged in the inner lumen of the puncture needle.

According to one advantageous configuration, the at least one optical component in the near-patient region fully seals the inner lumen of the puncture needle and the puncture needle has one or more lateral openings behind this sealing point, as seen from the near-patient end of the puncture needle. This has the advantage that, despite the region of the puncture needle sealed by the optical component, the liquid can be conveyed through the lateral openings lying behind from the near-patient region of the puncture needle through the puncture needle to the third grip section, and in particular its opening.

According to one advantageous configuration, the optical component is releasably coupled to the puncture needle, in particular coupled to the puncture needle with a form fit. The optical component may therefore be removed from the puncture needle and, for example, a guide wire may be introduced through the inner lumen of the puncture needle.

According to one advantageous configuration of the invention, the inner tubular body protrudes at the patient-remote end from the outer tubular body at least when the first, second and third grip sections are coupled to one another via the form-fit coupling elements. The patient-remote end of the inner tubular body in this case extends into the interior of the second grip section.

According to one advantageous configuration of the invention, the inner tubular body protrudes at the near-patient end from the outer tubular body at least when the first, second and third grip sections are coupled to one another via the form-fit coupling elements. The inner tubular body may in this case protrude from the outer tubular body at least by the length of the dilation section.

According to one advantageous configuration of the invention, the puncture needle protrudes at the patient-remote end from the inner tubular body at least when the first, second and third grip sections are coupled to one another via the form-fit coupling elements. The patient-remote end of the puncture needle in this case extends into the interior of the third grip section.

According to one advantageous configuration of the invention, the puncture needle protrudes at the near-patient end from the inner tubular body at least when the first, second and third grip sections are coupled to one another via the form-fit coupling elements. In this way, the medical puncture system in the basic state, in which the puncture system may for example be delivered by the manufacturer, can be used directly as an integrated tool for carrying out the entire puncture process. No special preparations need to be carried out on the puncture system by the user. Rather, the puncture system may be guided directly with the tip of the puncture needle forward to the desired location inside the body of the patient. This may, for example, be additionally checked and visualized by an ultrasound scan or radiological monitoring. Once the puncture needle is correctly placed, the individual parts of the puncture system may as already explained above then be successively displaced, or removed, until finally only the shaft unit remains on the patient. The length of the part of the puncture needle protruding from the third grip section may, for example, be at least 1.5 times as great as the length of the part of the outer tubular body protruding from the first grip section.

In this way, the puncture system according to the invention allows a kind of all-in-one puncture and correspondingly an optimized working practice of a nephrolitholapaxy. The operator does not need to change between different instruments.

According to one advantageous configuration of the invention, the puncture needle protrudes at the near-patient end from the inner tubular body with a free length which is at least 30% of the length of the outer tubular body, in particular at least 50% or 70% of the length of the outer tubular body. This applies particularly in an operating state of the medical puncture system in which the shaft unit, the dilator unit and the puncture needle unit are assembled with one another, for example when the first, the second and the third grip sections are coupled to one another via the form-fit coupling elements. The puncture needle therefore protrudes with its puncture needle tip relatively far out of the outer tubular body, which is favorable for carrying out a mini-PCNL.

According to one advantageous configuration of the invention, the puncture needle is at least 1.5 times or at least two times as long as the outer tubular body.

According to one advantageous configuration of the invention, the puncture system is adapted for carrying out a minimally invasive percutaneous nephrolitholapaxy (mini-PCNL). Likewise, the puncture system is equally advantageously suitable for comparable minimally invasive interventions, in particular for endoscopic interventions. Possible further fields of application may for example be general surgery, pediatric surgery, visceral surgery, trauma surgery or orthopedics, spine surgery and gynecology. Use in the area of thoracic surgery in the scope of video-assisted thoracoscopy (VATS) may also be envisioned. There may also be further fields of application in veterinary medicine. Other application possibilities may be envisioned in nonmedical areas, for example on or in pump and tube systems.

According to one advantageous configuration of the invention, the outer tubular body is length-adjustable and/or has at least one premade break point for selective shortening of the outer tubular body and/or is configured to be slitted in the longitudinal direction from the patient-remote end. This has the advantage that the outer tubular body can be adapted in respect of its length, directly while an operation is being carried out, to specific features of the operation and of the patient, i.e. it can be shortened to a desired length. The irrigation flow can thereby be optimized and the washing out of stone fragments improved, and the outer tubular body is prevented from standing out unnecessarily far from the patient, which would interfere with carrying out interventions. For example, the outer tubular body may be configured to be slitted over a section of from 10 to 50% of the total length starting from the patient-remote end. Likewise, the first grip section may be configured to be slitted so that the grip section can also be divided because of the slitted configuration. If there are premade break points, these may be arranged at equal or unequal spacings from one another over the longitudinal extent of the outer tubular body, for example at spacings of at least 1 cm.

In a further independent aspect, the invention relates to an outer tubular body in the form of an Amplatz shaft for carrying out percutaneous nephrolitholapaxy, wherein the Amplatz shaft has a plurality of circumferentially running notches distributed over its longitudinal extent, by which premade break points are formed for selective shortening of the Amplatz shaft at a respective point selected by the user. The advantages explained above may also thereby be achieved. The Amplatz shaft may have further one or more of the aforementioned features of the outer tubular body.

It is also possible to shorten the Amplatz shaft by means of a tool while a nephrolitholapaxy is being carried out, i.e. when it is already applied on the patient. This is advantageous in particular when the outer tubular body is configured as an Amplatz shaft for carrying out a percutaneous nephrolitholapaxy. For example, a pipe cutter may be used as the tool, especially when the material of the outer tubular body is relatively hard. If it is a softer material, shears may for example be used for the cutting. This procedure is possible even when the Amplatz shaft is formed without the premade break points explained above.

To this extent, the invention also relates to a method for adapting the length of an Amplatz shaft for carrying out a percutaneous nephrolitholapaxy, in which the Amplatz shaft is shortened to a desired extent before or while the percutaneous nephrolitholapaxy is carried out on the patient, for example by a portion being separated from the Amplatz shaft by means of a tool and/or a portion being broken off from the Amplatz shaft by using at least one premade break point.

### DESCRIPTION OF THE DRAWINGS

The invention is explained in more detail below with the aid of exemplary embodiments with the use of drawings, in which

FIG. 1 shows a medical puncture system in a perspective view,

FIG. 2 shows a detail enlargement of the near-patient region of the medical puncture system according to FIG. 1,

FIG. 3 shows individual structural units of the medical puncture system,

FIG. 4 shows the grip section of the medical puncture system in a partially cut away perspective view,

FIG. 5 shows the grip section according to FIG. 4 in a side view,

FIG. 6 shows a sectional view according to the section plane B-B represented in in FIG. 5,

FIG. 7 shows a sectional view according to the section plane A-A represented in in FIG. 5,

FIG. 8 shows a further partial sectional view similar to FIG. 4,

FIG. 9 shows a grip section according to FIG. 8 in a side view,

FIG. 10 shows a sectional view according to the section plane D-D represented in FIG. 9,

FIG. 11 shows a sectional view according to the section plane C-C represented in FIG. 9,

FIG. 12 shows a further partial sectional view similar to FIG. 4,

FIG. 13 shows the grip section according to FIG. 12 in a side view,

FIG. 14 shows a sectional view according to the section plane E-E represented in FIG. 13,

FIG. 15 shows an image reproduction unit,

FIG. 16-19 show steps when carrying out a mini-PCNL,

FIG. 20 shows a further embodiment of a shaft unit in a side view,

FIG. 21 shows the shaft unit according to FIG. 20 in a front view of the first grip section,

FIG. 22 shows the shaft unit according to FIG. 20 in a modified form,

FIG. 23 shows the placement of the shaft unit according to FIG. 22 on the patient,

FIG. 24-28 show a shaft unit with premade break points,

FIG. 29-33 show a shortened tubular body with a termination body.

### DETAILED DESCRIPTION

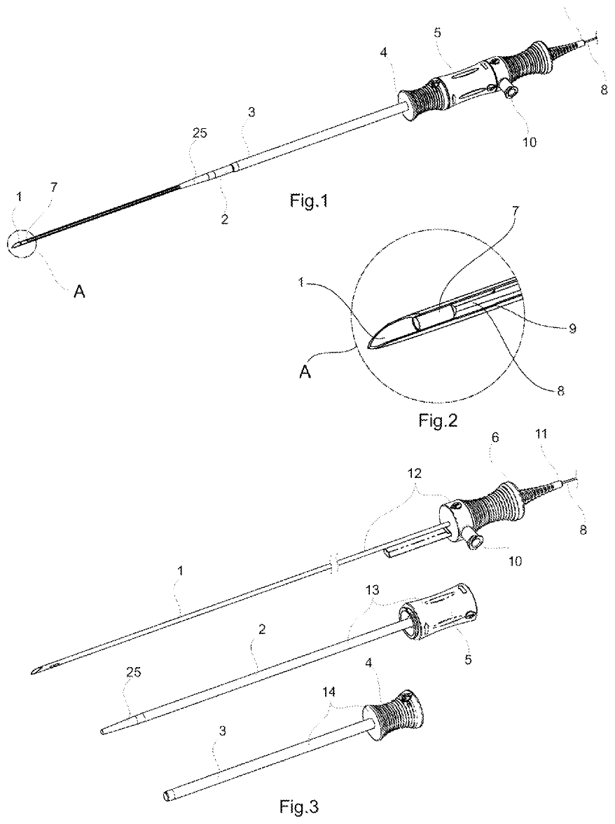

The basic structure of the medical puncture system will initially be explained with reference to FIGS. 1 to 3. The medical puncture system is essentially constructed from three structural units, namely a shaft unit 14, a dilator unit 13 and a puncture needle unit 12. The puncture needle unit 12 has a puncture needle 1 and a third grip section 6 connected to the puncture needle 1. The dilator unit 13 has an inner tubular body 2 and a second grip section 5, which is connected to the inner tubular body 2. The shaft unit 14 has an outer tubular body 3 and a first grip section 4 connected thereto. The puncture needle 1 may be tipped, i.e. it may have a puncture needle tip, in the near-patient region A, i.e. at the end. The puncture needle 1 may be configured as a hollow needle. In this case, optics 7 can be arranged inside the puncture needle. The optics 7 may be fed out via an electrical cable 8 or light guide through the puncture needle 1 and through the third grip section 6 and connected to an image reproduction unit. At the patient-remote end, for example behind the third grip section 6, the puncture needle unit 12 may have kink protection 11 by which kinking of the electrical cable 8 or of the light guide is avoided. In addition, the puncture needle 1 may have one or more side openings 9 which are arranged behind the optics 7, as seen from the near-patient end of the puncture needle 1. Through the side openings 9, body fluids can enter into the interior of the puncture needle 1. The puncture needle unit 12 may for example have an opening 10 in the region of the third grip section 6, for example with a Luer connection, in order for liquids received via the side openings 9 to be conveyed outward through it and collected and/or analyzed.

In the dilator unit 13, it can be seen that the outer tubular body 2 has a dilation section 25 at the near-patient end. In the delivery state which is represented in FIG. 1, the second grip section is located behind the first grip section and the third grip section is located behind the second grip section 5, as seen from the near-patient end A. The inner tubular body 2 protrudes at the near-patient end from the outer tubular body 3 at least by the length of the dilation section 25. Further, the puncture needle 1 protrudes at the near-patient end substantially further from the inner tubular body 2, for example by at least 1.5 times the length of the inner tubular body.

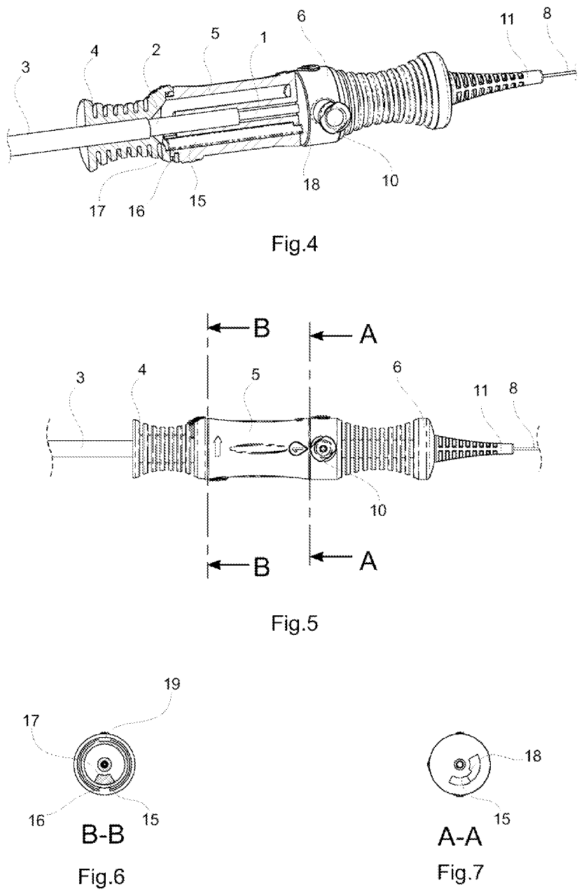

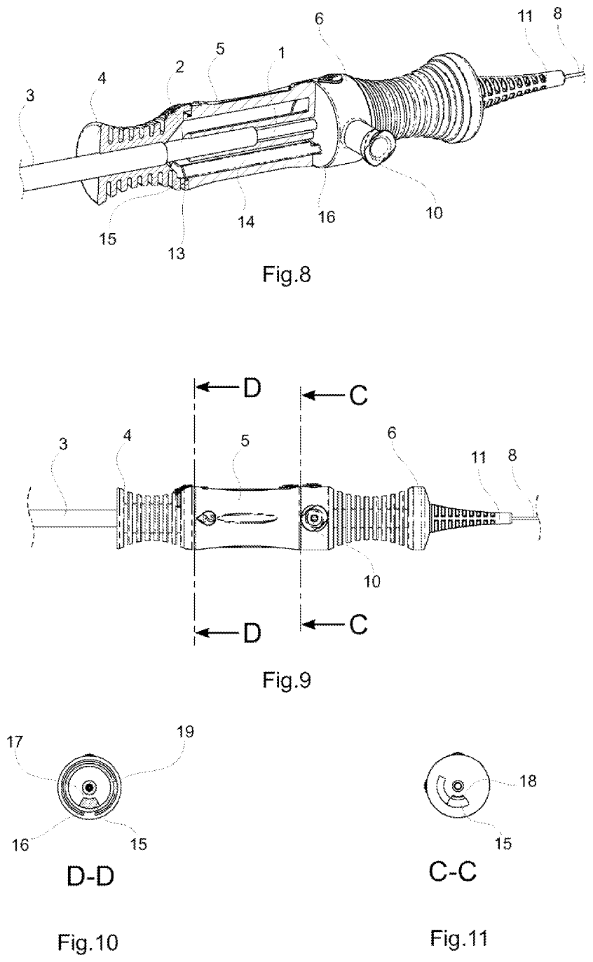

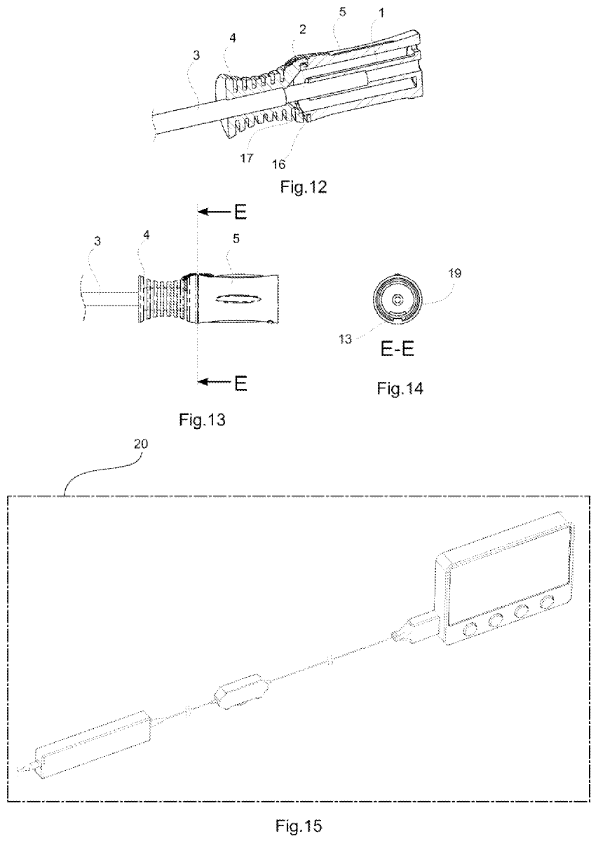

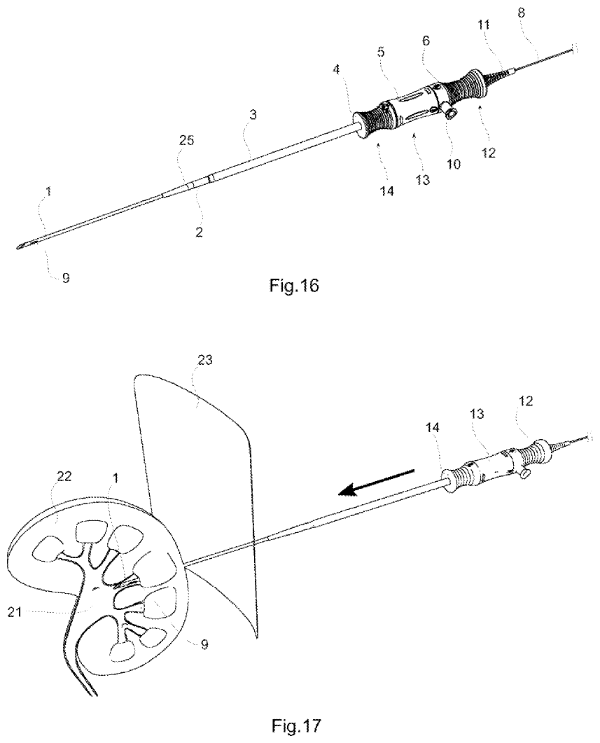

The releasing of the grip sections 4, 5, 6 from one another and the sequence of this release, which is established by the securing mechanism, are to be explained with the aid of FIGS. 4 to 14.

In the initial setting (FIG. 4), all grip sections 4, 5, 6 are connected to one another with a form fit. The connections can be released only by a rotation of the dilator unit 13 in the region of the second grip section 5. The shaft unit 14 is connected form-fittingly by a form-fit coupling element in the form of an Amplatz bar 16, which is located on the first grip section 4, to the dilator unit 13 in the region of the second grip section 5. A securing element in the form of a puncture needle lock 15 on the third grip section 6 engages into an Amplatz groove 17 on the first grip section 4 and thereby prevents a possible rotating movement of the first grip section 4 relative to the second grip section 5. This ensures that the shaft unit 14 cannot be released from the dilator unit 13 and pushed forward, so long as this is blocked by the securing element.

The sequence of the individual process steps is made simpler for the user by clear labeling and/or markings, for example arrows, numbering, or the individual grip sections 4, 5, 6.

In the puncture system according to the invention, the skin 23 is initially punctured, for example under ultrasound monitoring. The puncture needle 1 may have a particular surface structure on the distal region for better ultrasound visibility, for example sand blasting, coating, etc. FIG. 17 shows the position of the puncture needle in the calyceal system 21 of the kidney 22. In this position, visual assessment now additionally takes place. Furthermore, urine is received via the side openings 9 on the distal region of the puncture needle 1 and discharged via a through-opening 10 on the third grip section 6. This through-opening 10 may have a standardized connection, for example a Luer lock connection, through which for example contrast agent can be delivered, for example by syringes or tubes.

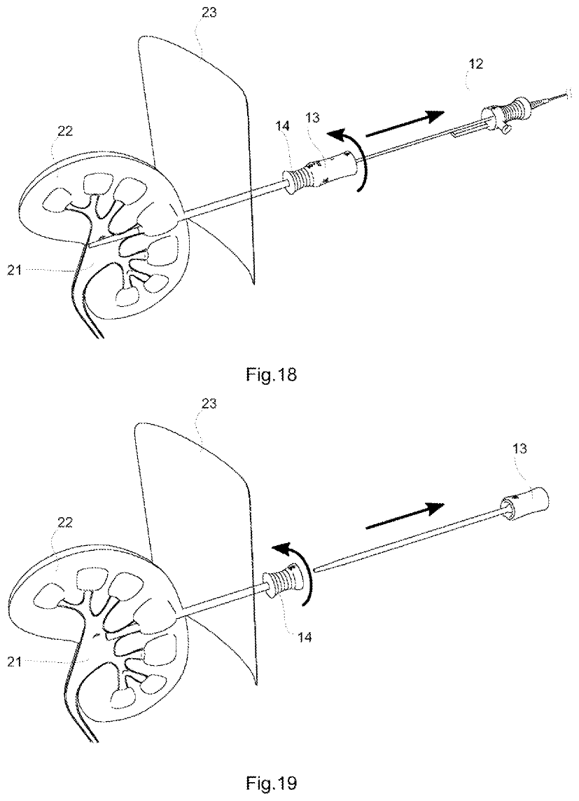

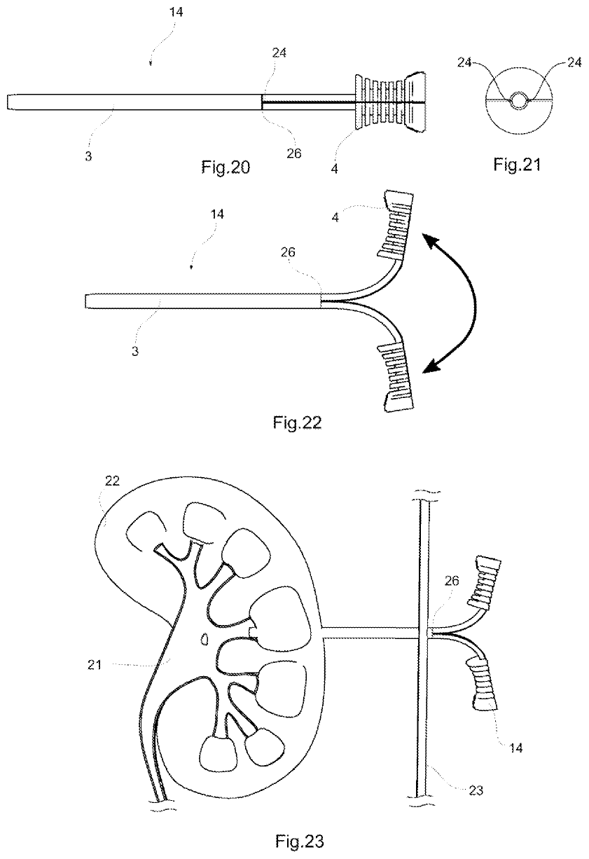

When the puncture needle tip has the correct position in the calyceal system 21 of the kidney 22, the dilator unit 13 is rotated as far as a perceptible stop. FIG. 9 shows the representation when the dilator unit 13 is rotated through 90°. The sectional representation C (FIG. 11) shows the opened position. In this position, the dilator unit 13 is freed by the dilator groove 19 and can then be released from the puncture needle unit 12. The dilator unit 13 is subsequently pushed forward together with the shaft unit 14 axially over the puncture needle 1 to beyond the optics 7. When the position of the dilator unit 13 according to FIG. 18 is reached, the puncture needle unit 12 is removed.

The dilator unit 13 is subsequently rotated further as far as a perceptible stop. The sectional representation E-E in FIG. 14 shows the opened position. The shaft unit 14 is in this position freed by the dilator groove 19 and can be released from the dilator unit. The shaft unit 14 is subsequently pushed forward axially beyond the dilator unit 13 into the calyceal system 21 of the kidney 22. When the position of the shaft unit 14 is correct, the dilator unit 13 is subsequently removed.

FIG. 15 shows by way of example an image reproduction unit 20, which may be attached to the electrical cable 8 or the light guide. The image reproduction unit 20 may for example have an image display device (monitor), an interface and a dimmer. Located on the distal region of the puncture needle 1, as mentioned, there are optics 7, or in general at least one optical component, which may be configured as a small camera and an integrated light unit, by which the near-puncture region can be visually observed by the user. Via a near-user cable 8, for example, the puncture system may be connected to the image reproduction unit 20. The cable 8 may be fed out centrally from the third grip section 6 so that the puncture system can be rotated without problems and operated with both hands.

In the puncture system according to the invention, a guide wire is no longer necessary since the position of the puncture needle with the tip can be visually assessed. The X-ray exposure of the patient and staff is therefore also less, and may be entirely obviated.

An optimal irrigation property may be achieved by a minimized shaft length of the outer tubular body 3. This may be implemented by individual adjustment of the shaft length. According to one advantageous configuration of the invention, the outer tubular body 3 may be configured with notches 24 at the patient-remote end. The notches 24 run axially outside the outer tubular body 3 with a defined notch length. The notches make a premade break point possible, so that when required the shaft can be split in order to enable an improved flow length. The representations of FIG. 22 und FIG. 23 show the outer tubular body with a split patient-remote end. There may additionally be a circumferential premade break point 26, for example at the end of the longitudinally running notch 24, at which the split parts of the shaft can be broken off.

A further possible configuration is a length-adjustable outer tubular body having assembled pipe parts. In the patient-remote region of the outer tubular body, the individual pipe parts have screw connections. The shaft length can be shortened by releasing the screw connection of the individual pipe parts.

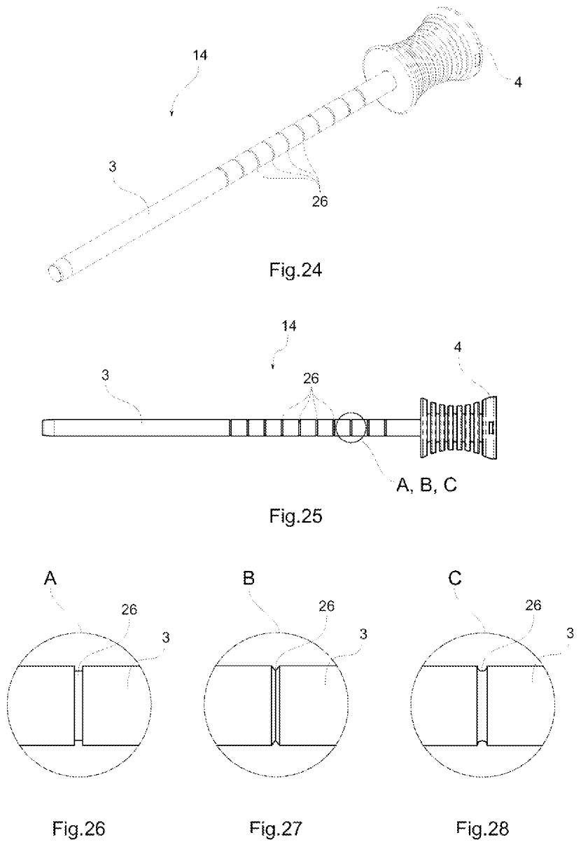

A further configuration is an outer tubular body 3 having a plurality of premade break points 26 running around on the circumference, as represented in FIGS. 24 to 28. A particular length section of the outer tubular body 3 may for example be provided with a plurality of such premade break points 26, which are arranged at equal or unequal spacings from one another, for example at spacings of 1 cm. The premade break points 26 may for example be configured as circumferential indentations (grooves) formed on the outer side, different profile cross sections of the indentations being conceivable. FIG. 26 shows a premade break point 26 with a rectangular cross section, FIG. 27 shows a premade break point 26 with a triangular cross section and FIG. 28 shows a premade break point 26 with a semicircular cross section. The shaft length of the outer tubular body 3 may be shortened by breaking at the desired premade break point 26 when the outer tubular body 3 is placed on the patient.

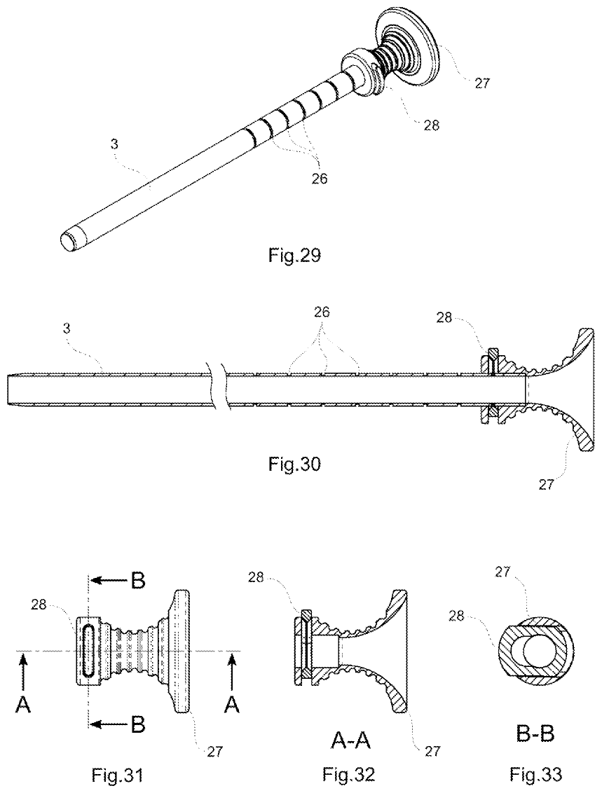

FIGS. 29 and 30 show the outer tubular body 3 according to FIGS. 24 to 28 in a form shortened at a premade break point 26. In addition, at the point where the outer tubular body 3 is shortened at a premade break point, a termination body 27 is fitted there. In principle, the shortened outer tubular body 3 may also be used further without such a termination body. By fitting the termination body 27, however, the exposed premade break point can be covered. With the termination body 27, better manual operation may possibly also be carried out on the remaining part of the outer tubular body 3.

It can be seen that the termination body 27 may have a funnel shape at least in an inner hollow region. In an end region of the termination body 27, which can be fitted externally onto the end section of the outer tubular body 3, the termination body 27 may have a fastening element 28. The fastening element 28 may in particular be configured to engage into a notch-like premade break point 26, so as to create a form-fit coupling of the termination body 27 to the outer tubular body 3. For example, the fastening element 28 may be configured as a displaceably arranged fastening clip which then engages by displacement into a notch-like premade break point 26.

Shortening of the outer tubular body by a telescopic apparatus may also be envisioned.

According to one advantageous configuration of the invention, the inner tubular body and/or the outer tubular body may consist of at least one hydrophilic material and/or be coated with at least one hydrophilic material.

According to one advantageous configuration of the invention, the outer tubular body may also be formed from plastic or at least one metallic material or a corresponding alloy, or be coated with at least one such material, over a partial length, the predominant length or the entire length. Furthermore, the outer tubular body may be configured in two parts from plastic and metal.

According to one advantageous configuration, the inner tubular body and/or the outer tubular body may have a conical or curved tip.

### LIST OF REFERENCE SIGNS

1 puncture needle
2 inner tubular body (e.g. dilator)
3 outer tubular body (e.g. Amplatz shaft)
4 first grip section (e.g. Amplatz grip)
5 second grip section (e.g. dilator grip)
6 third grip section (e.g. puncture needle grip)
7 optics
8 cable/light guide
9 needle side openings
10 opening optionally with Luer connection
11 kink protection
12 puncture needle unit
13 dilator unit
14 shaft unit
15 securing element
16 Amplatz bar
17 Amplatz shaft groove
18 dilator bar
19 dilator groove
20 image reproduction unit (interface, dimmer, monitor)
21 calyceal system
22 kidney
23 skin
24 notch
25 dilation section
26 premade break point
27 termination body
28 fastening element

1. A medical puncture system, comprising:
a shaft unit;
a dilator unit; and
a puncture needle unit configured for carrying out a puncture on a patient;
wherein the shaft unit comprises
an outer tubular body with an inner working lumen, and
a first grip section adapted for manual handling of the shaft unit, wherein the first grip section is connected at a patient-remote section to the outer tubular body,
wherein the dilator unit comprises
an inner tubular body with an inner puncture lumen, and
a second grip section adapted for manual handling of the dilator unit, wherein the second grip section is connected at a patient-remote section to the inner tubular body
wherein the inner tubular body is configured to be guidable through the inner working lumen of the outer tubular body of the shaft unit
wherein the puncture needle unit comprises
a puncture needle, and
a third grip section adapted for manual handling of the puncture needle unit, wherein the third grip section is connected at a patient-remote section to the puncture needle,
wherein the puncture needle is configured to be guidable through the puncture lumen of the inner tubular body of the dilator unit.

2. The medical puncture system as claimed in claim 1, wherein the first grip section is releasably coupled via a first at least one form-fit coupling element to the second grip section by a first form fit and/or the second grip section is releasably coupled via a second at least one form-fit coupling element to the third grip section by a second form fit.

3. The medical puncture system as claimed in claim 2, wherein the first grip section, the second grip section, and the third grip section together form a homogeneous grip section for gripping and manually handling the puncture system when they are coupled to one another via the first at least one form-fit coupling element and the second at lease one form-fit coupling element.

4. The medical puncture system as claimed in claim 2, wherein the second grip section is coupled by the first a form fit to the first grip section and is releasable from the first grip section by a first rotational movement in relation thereto and/or the third grip section is coupled by the second form fit to the second grip section and is releasable from the second grip section by a second rotational movement in relation thereto.

5. The medical puncture system as claimed in claim 1 wherein the puncture needle is longitudinally displaceable in relation to the inner tubular body of the dilator unit, and wherein, the puncture needle unit is configured to be withdrawable from the dilator unit in a direction facing forward from a the near-patient end of the medical puncture system.

6. The medical puncture system as claimed in claim 1 wherein the inner tubular body of the dilator unit is configured to be longitudinally displaceable in relation to the outer tubular body of the shaft unit, and wherein, the dilator unit is configured to be withdrawable from the shaft unit in a direction facing forward from a near-patient end of the medical puncture system.

7. The medical puncture system as claimed in claim 6, further comprising a securing mechanism configured to ensure that the dilator unit is withdrawable from the shaft unit only when the third grip section is decoupled from the second grip section and/or the puncture needle unit is withdrawn from the dilator unit.

8. The medical puncture system as claimed in claim 7, wherein a first form-fit coupling between the first grip section and the second grip section is secured via the securing mechanism, and wherein the securing mechanism has a secured state and an unsecured state, wherein release of the first grip section from the second grip section is prevented in the secured state and release of the first grip section from the second grip section is enabled in the unsecured state.

9. The medical puncture system as claimed in claim 8, wherein the puncture needle unit comprises has at least one securing element which blocks the securing mechanism in the secured state which prevents the securing mechanism from being put into the unsecured state.

10. The medical puncture system as claimed in claim 1 wherein the inner tubular body comprises a dilation section in a near-patient region which tapers on an outer circumference of the dilation section toward the near-patient end of the medical puncture system.

11. The medical puncture system as claimed in claim 1 wherein the puncture needle is configured as a hollow needle comprising an opening in a region of the third grip section wherein the opening in the hollow needle is configured so that liquid is conveyable outward through the puncture needle from a near-patient region of the puncture needle.

12. The medical puncture system as claimed in claim 1 wherein the puncture needle comprises has at least one optical component in a near-patient region through which light is directable toward a patient and/or through which images are recordable.

13. The medical puncture system as claimed in claim 12, wherein the at least one optical component is coupled releasably to the puncture needle and/or with a form fit to the puncture needle.

14. The medical puncture system as claimed in claim 1 wherein the inner tubular body of the dilator unit protrudes at a near-patient end from the outer tubular body of the shaft unit when the first grip section, the second grip section, and the third grip section are coupled to one another via form-fit coupling elements.

15. The medical puncture system as claimed in claim 1 wherein that the puncture needle protrudes at a near-patient end from the inner tubular body of the dilator unit when the first grip section, the second grip section, and the third grip section sections are coupled to one another via form-fit coupling elements.

16. The medical puncture system as claimed in claim 1 wherein the medical puncture system is adapted for carrying out a minimally invasive percutaneous nephrolitholapaxy (mini-PCNL).

17. The medical puncture system as claimed in claim 1 wherein the outer tubular body of the shaft unit is length-adjustable and/or has at least one premade break point for selective shortening of the outer tubular body and/or is configured to be slitted in a longitudinal direction from a patient-remote end.

18. An Amplatz shaft for carrying out percutaneous nephrolitholapaxy, comprising a plurality of circumferentially running notches distributed over a longitudinal extent of the Amplatz shaft, wherein the plurality of circumferentially running notches include premade break points formed for selective shortening of the Amplatz shaft at a respective point of the premade break points selectable by a user.
